(19) United States
(12) Patent Application Publication
(10) Pub. No.: US 2003/0109921 A1
(43) Pub. Date: Jun. 12, 2003

(54) System for implanting prosthetic heart valves

(76) Inventors: Riyad Moe, Austin, TX (US); John Kenneth Ryder, Round Rock, TX (US)

Correspondence Address: Timothy L. Scott, Senior Intellectual Property Counsel, SULZER MEDICA USA INC., 3 East Greenway Plaza, Suite 1600, Houston, TX, 77046, US

(21) Appl. No.: 10/014,091

(22) Filed: Dec. 11, 2001

(51) Int. Cl. A61F 2/24

(52) U.S. Cl. 623/2.11; 623/2.41

(57) ABSTRACT

A heart valve having a sewing ring marked by six radially spaced marks. The marks may be spaced at regular intervals around the sewing ring, or they may correspond to asymmetric features of the valve, such as commissures supporting differently sized leaflets. Some of the marks may be placed at a midpoint between adjacent commissures. A template for temporary placement at an implantation site within the patient's heart has six radially spaced marks, corresponding to the marks on the sewing ring.

### BACKGROUND

1. Technical Field

The present invention pertains to prosthetic heart valves and in particular to apparatus and methods for implanting prosthetic heart valves.

2. Background of the Invention

Ever since 1950, when blood oxygenators made open heart surgery feasible, it has been possible to treat some forms of heart disease by replacing one of the patient's heart valves with a prosthetic valve. A prosthetic heart valve is implanted into an annular opening in a heart created when the diseased valve is removed. Early heart valve prostheses included ball-and-cage valves and disc-and-cage valves in which a ball or a disc was housed in a cage. One side of the cage provided an orifice through which blood flowed either into or out of the heart, depending on the valve being replaced. When blood flowed in a forward direction, the energy of the blood flow forced the ball or disc to the back of the cage allowing blood to flow through the valve. When blood attempted to flow in a reverse direction, or “regurgitate,” the energy of the blood flow forced the ball or disc into the orifice in the valve and blocked the flow of blood.

A “mechanical” valve is another type of prosthesis comprising an annular valve body in which one, two, or three leaflet occluders are pivotally mounted. The occluders are typically substantially rigid. The occluders move between a closed position, in which the leaflets are mated and block blood flow in the reverse direction, and an open position, in which the occluders pivot away from each other and do not block blood flow in the forward direction. The energy of blood flow causes the occluders to move between their open and closed positions.

The leaflets of “tissue” valves are usually flexible and made from tissue, such as specially treated porcine or bovine pericardial tissue. A tri-leaflet tissue valve comprises an annular valve body in which three flexible leaflets are mounted to a supporting portion of the valve body, called a “stent,” located at the circumference of the annulus. When blood flows in the forward direction, the energy of the blood flow deflects the three leaflets away from the center of the annulus and allows blood to flow through. When blood flows in the reverse direction, the three leaflets engage each other in a coaptive region, occlude the valve body annulus, and prevent the flow of blood.

Heart valves made from a man-made material such as polyurethane or another biocompatible polymer may have two or three leaflets and may have a stent to increase the structural strength of the valve while allowing the leaflets to remain flexible. “Polymeric” valves may be sutured directly to the site of an explanted heart valve, or a sewing ring may be sutured to the valve body and sutures attaching the valve to the heart may pass through the sewing ring.

Each of these types of valves must be secured within the patient's heart at the site of an explanted natural valve. Typically, a surgeon has placed multiple sutures through a sewing ring on the prosthetic valve and through the tissue of the heart. When all the sutures had been placed through both the prosthetic valve and the tissue, the valve is advanced along the sutures to the implantation site and the sutures are tied. If, however, the placement of the sutures in the tissue and in the sewing ring does not coincide, the tissue would either pucker or be stretched between adjacent sutures. Surgeons attempt to minimize this undesirable condition by observing physical features at the implant site and attempting to correlate those features to features on the prosthetic valve. Such features might include the commissures or from one to four marks on the sewing ring of the prosthetic valve. There remains a need, however, to provide additional assistance to surgeons for accurately implanting prosthetic heart valves.

### SUMMARY OF THE INVENTION

The invention provides additional aid to a surgeon implanting a prosthetic heart valve by providing a heart valve having a sewing ring marked by at least six radially spaced marks. The marks may be spaced at regular intervals around the sewing ring, or they may correspond to asymmetric features of the valve, such as commissures supporting differently sized leaflets. Some of the marks may be placed at a midpoint between adjacent commissures. Six sutures are more effective than a lesser number of sutures and is a desirable number of sutures for minimizing blood leakage around the sewing cuff without excessive complications during insertion. Nevertheless, prior valves have had sewing rings with between one and four marks, requiring a surgeon to estimate placement of the sutures around both the sewing ring and around the implantation site in the heart.

A template is provided for placement at an implantation site within the patient's heart. The template may be a sizing device useful in determining the correct size prosthetic heart valve to be implanted. The template has six radially spaced marks, corresponding to the marks on the sewing ring. In using the apparatus of the invention, the template is placed within the heart at the site of an explanted valve. The surgeon orients the template in a desired position and places suture from the heart to the sewing ring of the valve. The sutures are placed carefully from positions adjacent the marks on the template to corresponding marks on the sewing ring. After the sutures are in place, the heart valve can be advanced along the sutures to the implantation site. Because the sutures can be placed accurately in corresponding locations on both the heart valve and in the heart tissue, stretching and puckering of the tissue is eliminated or substantially reduced when the sutures are tightened and tied.

It is an object, therefore, of the present invention to provide a prosthetic heart valve having a sewing ring with six marks for guiding placement of sutures.

It is another object of this invention to provide a set of apparatus comprising a heart valve with six marks on a sewing ring and a template having corresponding marks for guiding placement of sutures in an implantation site.

These and other objects and features of the invention will be apparent from the following detailed description, made with reference to the accompanying drawings.

### BRIEF DESCRIPTION OF THE DRAWINGS

FIG. 1 is a perspective view of a mechanical heart valve with a sewing ring.

FIG. 2 is a top view of the mechanical heart valve of FIG. 1.

FIG. 3 is a perspective view of a biologic heart valve with a sewing ring.

FIG. 4 is a top view of the biologic heart valve of FIG. 3.

FIG. 5 is a perspective view of a template for use with the heart valves of either FIG. 1 or FIG. 3.

FIG. 6 is a top view of the template of FIG. 5.

FIG. 7 is view of a heart with a heart valve and template.

### DESCRIPTION OF THE PREFERRED EMBODIMENTS

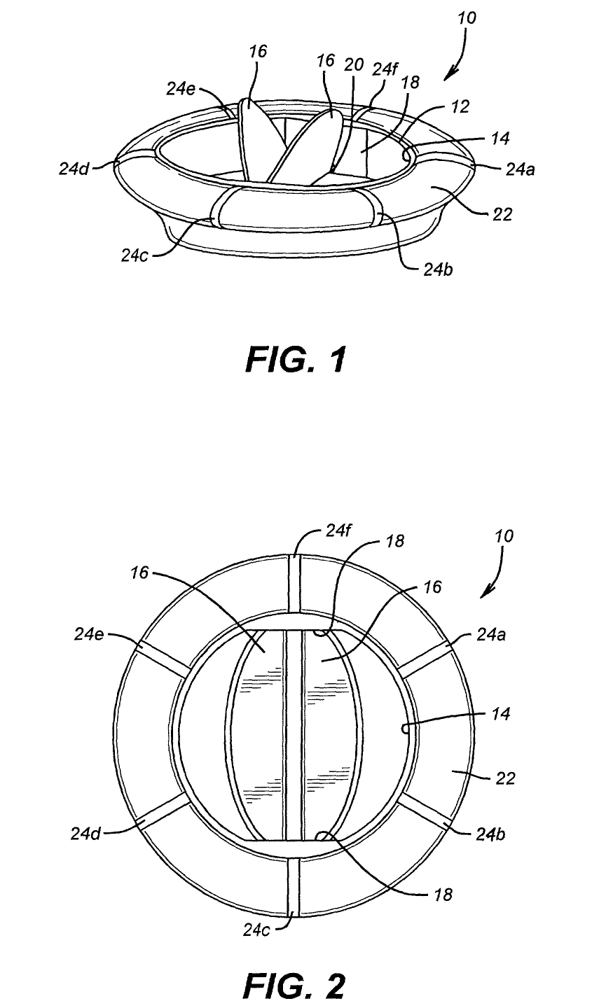

A mechanical heart valve 10, as illustrated in FIG. 1, usually has an annular valve body 12. The valve body 12 is most frequently formed of so-called pyrolytic carbon, but may also be made of metal, ceramic, or other hard, wear-resistant, biocompatible substance. The valve body 12 has an inner wall 14 that is substantially circular, but interrupted by two opposed, parallel flats 18. Two occluders 16 are mounted between the flats and pivot around pins or ears (not shown) that are mounted in recesses 20. The occluders 16 move between an open position, as shown, and a closed position, alternately permitting and resisting the flow of blood through the valve. A fabric sewing ring 22 circumferentially surrounds the valve body 12. The sewing ring 22 and the valve body 12 are connected, usually by a stiffening ring (not shown), as is well known in the art. The annular valve body may or may not rotate in the stiffening ring. Six marks 24a, 24b, 24c, 24d, 24e and 24f are provided circumferentially on the sewing ring. These marks are preferably symmetrically spaced around the sewing ring 22. The six marks allow a surgeon to place sutures around the sewing ring more accurately by placing a suture at each of the marks rather that estimating the placement of the sutures, as was done in the past.

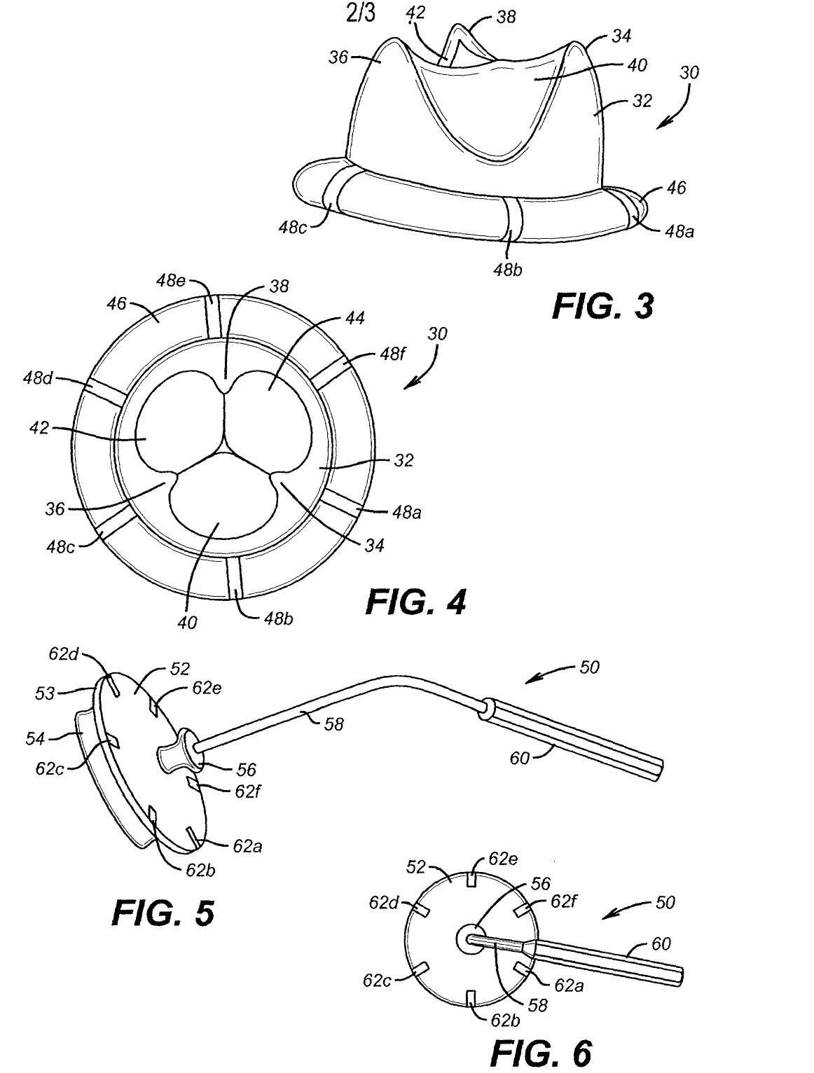

The invention may also be used with other types of heart valves. A flexible leaflet valve 30 is illustrated, for example, in FIG. 3 and FIG. 4. The flexible leaflet valve 30 comprises a valve body 32 with a plurality of commissure supports 34, 36, 38 surrounding a central orifice. Flexible leaflets 40, 42, 44, connected between adjacent commissure supports, open and close to permit and resist the flow of blood through the valve 30. The leaflets are illustrated in a closed position. The leaflets may be comprised of treated tissue, such as bovine or porcine pericardium, or of an artificial material, such as an elastomeric polymer. The flexible leaflet valve 30 may also have a supporting stent (not shown) as is known in the art. A circumferential sewing ring 46 surrounds the valve body 32 and is connected to the valve body. Six marks 48a, 48b, 48c, 48d, 48e and 48f are provided circumferentially on the sewing ring. As explained above, these marks may be symmetrically spaced around the ring. They may also be placed to correspond with features of the valve body 32. For example, if the leaflets 40, 42, 44 are of different sizes, the commissure supports 34, 36, 36 would be separated by different distances. Three marks 48a, 48c and 48e could be placed adjacent respective commissure supports 32, 36, 38. The remaining three marks 48b, 48d, 48f could bisect the distance between adjacent marks, for example, mark 48b could be midway between mark 48a and 48c.

A template 50 is provided with circumferential marks 62a, 62b, 62c, 62d, 62e, 62f to correspond with the particular valve 10 or 30 being implanted. The template may also be used as a sizing device to help the surgeon select the proper size valve for implantation. The template 50 comprises a disc 52 having an edge 53 corresponding to the shape of the sewing ring 22 or 46. The circumferential marks 62a, 62b, 62c, 62d, 62e, and 62f are preferably next to the edge 53. The template 50 may also have a prominence 54 on one side of the disc 52. The prominence is shaped to correspond generally to the shape of the valve body 12 or 32 such that the prominence may be inserted into an implantation site in the heart, allowing the surgeon to determine if a particular valve body will fit into the site. A coupling 56 on another side of the disc 52 connects the disc 52 to a bendable shaft 58. The bendable shaft 58 connects to a handle 60.

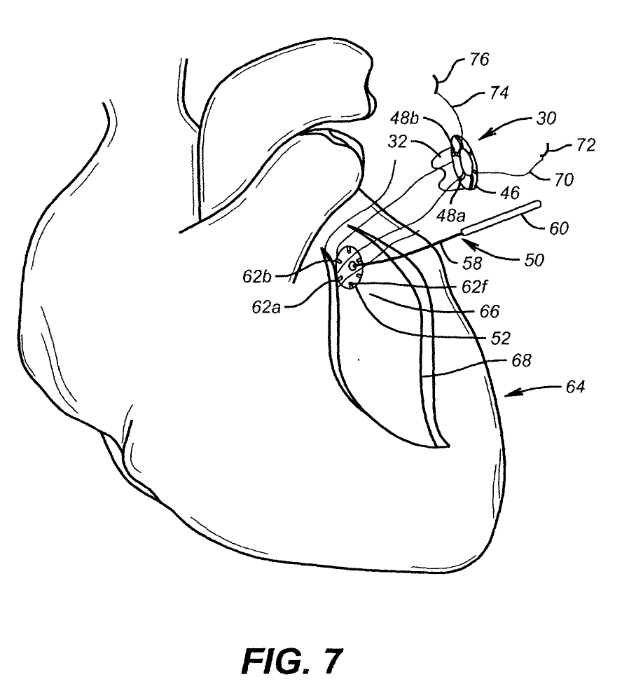

As shown in FIG. 7, the set of apparatus comprising a heart valve 30 and template 50 is used when implanting the heart valve 30 in a patient's heart 64. With the heart 64 stopped, an implantation site 66 is exposed through an incision 68. A diseased natural valve will be excised from the implantation site and the template 50 positioned at the implantation site. The marks 62 on the template are oriented in an optimum location in view of the surrounding physical structure of the heart. Sutures 70, 74 with attached needles 72, 76 are drawn through the tissue of the heart adjacent the marks 62 on the template 50 and then are drawn through the sewing ring 46 at corresponding marks 48 on the sewing ring. When sutures have been placed at all six marks 62 on the template and connected to all six corresponding marks 48 on the sewing ring 30, the valve 30 can be advanced along the sutures to the implantation site 66 and the sutures can be tied to secure the valve. The incision 68 can be closed and the heart can again begin pumping blood throughout the body of the patient.

The foregoing describes preferred embodiments of the invention and is given by way of example only. The invention is not limited to any of the specific features described herein, but includes all variations thereof within the scope of the appended claims.

What is claimed is:

1. A set of medical apparatus comprising
a prosthetic heart valve comprising a sewing ring, said sewing ring having a first set of six or more marks circumferentially placed on said sewing ring and
a template generally corresponding in shape to said sewing ring and having a second set of six or more marks circumferentially placed thereon, the placement of said second set of marks on said template corresponding to the placement of said first set of marks on said sewing ring.

2. The set of claim 1 wherein said first set of marks are evenly spaced around an outer edge of said sewing ring.

3. The set of claim 2 wherein said heart valve is a mechanical valve.

4. The set of claim 2 wherein said heart valve has flexible leaflets.

5. The set of claim 4 wherein said heart valve has polymeric leaflets.

6. The set of claim 4 wherein said heart valve has tissue leaflets.

7. The set of claim 1 wherein said first set of marks are not all equally spaced around said outer edge.

8. The set of claim 7 wherein said heart valve has flexible leaflets and a plurality of commissure supports and at least some of said six marks of said first set of six marks are aligned with said commissure supports.

9. A prosthetic heart valve comprising a sewing ring, said sewing ring having six or more marks circumferentially placed on said sewing ring.

10. The heart valve of claim 9 wherein said six marks are evenly spaced around an outer edge of said sewing ring.

11. The heart valve of claim 10 wherein said heart valve is a mechanical valve.

12. The heart valve of claim 10 wherein said heart valve has flexible leaflets.

13. The heart valve of claim 12 wherein the heart valve has polymeric leaflets.

14. The heart valve of claim 12 wherein said heart valve has tissue leaflets.

15. The heart valve of claim 9 wherein said six marks are not all equally spaced around an outer edge of said sewing ring.

16. The heart valve of claim 15 wherein said heart valve has flexible leaflets and a plurality of commissure supports and at least some of said six marks are aligned with said commissure supports.
